(19) United States
(12) Patent Application Publication
(10) Pub. No.: US 2024/0198097 A1
(43) Pub. Date: Jun. 20, 2024

(54) DEVICES AND METHODS FOR TREATING INFECTED TISSUE

(71) Applicant: NANOVIS, LLC, Columbia City, IN (US)

(72) Inventors: David A. DETWILER, West Lafayette, IN (US); Alan M. KRAFT, Lafayette, IN (US); James T. MCCARTHY, West Lafayette, IN (US); Gordon P. KISTLER, Fort Wayne, IN (US)

(73) Assignee: NANOVIS, LLC, Columbia City, IN (US)

(21) Appl. No.: 18/588,877

(22) Filed: Feb. 27, 2024

Related U.S. Application Data
(63) Continuation of application No. PCT/US22/75628, filed on Aug. 30, 2022.
(60) Provisional application No. 63/238,626, filed on Aug. 30, 2021.

(51) Int. Cl. A61N 1/32; A61F 2/28; A61F 2/30; A61F 13/00; A61K 31/155; A61K 41/00; A61M 1/00; A61M 5/32; A61N 1/04; A61N 1/36

(52) U.S. Cl. CPC A61N 1/327; A61F 2/30; A61F 13/00063; A61K 31/155; A61K 41/0047; A61M 1/90; A61M 5/3286; A61N 1/0416; A61N 1/36034; A61F 2002/2821; A61M 2205/054

(57) ABSTRACT

A system for treating infected tissue includes an applicator device. The applicator device has an electrode pair spaced apart and is positioned against infected tissue. An electrical power supply to generate a pulsed electric field across the electrode pair, resulting in the application of a pulsed electric field through the infected tissue. The pulsed electric field has a field strength within a range that when the pulsed electric field is applied, pores are formed in membranes of cells of the infected tissue. A method for treating infected tissue is disclosed. The method includes positioning an electrode pair against the infected tissue; applying a pulsed electric field across the electrode pair. The pulsed electric field has a field strength that when applied, pores are formed in membranes of cells of the infected tissue; and applying an antimicrobial agent into or onto the infected tissue when the pulsed electric field is applied.

### CROSS-REFERENCE TO RELATED APPLICATIONS

This application is a continuation of International Application No. PCT/US2022/075628 filed Aug. 30, 2022, and entitled “Devices and Methods For Treating Infected Tissue”, which claims priority benefit under 35 U.S.C. § 119(e) to U.S. Provisional Application No. 63/238,626, filed Aug. 30, 2021, entitled, “Devices and Methods To Treat Localized Infections,” the contents of both are incorporated herein in their entirety by reference.

### FIELD OF THE INVENTION

The present disclosure relates to devices and methods for treating infected tissue. More specifically, the disclosure relates to reversible electroporation devices and methods for treating infected human tissue.

### BACKGROUND OF THE INVENTION

Electric pulses have been applied to various cells and microbes for a number of reasons. For example, methods of applying electric pulses combined with antibiotic compounds have been used to reduce. or destroy, a number of viable microbes. Examples of such methods are described in U.S. patent No.: U.S. Ser. No. 11/123,428, filed on Aug. 12, 2020, which is incorporated herein by reference in its entirety.

Also, by way of example, methods of applying electric pulses have been used to stimulate the proliferation and differentiation of Eukaryotic cells. Examples of such methods are described in U.S. Patent Application Publication No.: US 20210340520, filed on Jul. 7, 2021, which is also incorporated herein by reference in its entirety.

Another method of applying electric pulses to cells is known as electroporation. Electroporation is a technique in which an electrical field is applied to cells in order to form pores in the cell membrane and, therefore, increase the permeability of the cell membrane to such things as various drug treatments, plasmids, DNA or the like.

However, using electroporation techniques for the treatment of localized infections has proven to be problematic. This is because electroporation applied with high field strengths (for example, higher than 40 kV/cm) can often cause permanent damage to the host tissue. Alternatively, electroporation applied with lower field strengths (for example, less than 30 kV/cm) may produce resealable pores and cause far less damage to the host tissue. However, the pores induced by such electroporation methods alone are often of insufficient quantity or size to enable sufficient diffusion of drugs across the membranes of the infected cells to effectively treat the infected cells.

Accordingly, there remains a need to improve electrical pulsing apparatus and methods for the treatment of infected cells. In particular, there is a need to improve electroporation apparatus and methods to effectively treat in-vivo localized infections of a patient.

The present disclosure is directed to overcoming these and other deficiencies in the art.

### SUMMARY OF THE INVENTION

The present disclosure offers advantages and alternatives over the prior art by providing an electroporation system for treating localized infections. The electroporation system applies an electric field having a field strength that induces reversible pore formation across target infected tissue. The electroporation system also includes an antimicrobial agent, which operates to cross or integrate into the membranes of infected cells when the electric field is applied to the infected tissue. The pores opened by the electric field combined with the antimicrobial agent enables drug agents (e.g., antibiotic, antiseptic or antifungal agents) to diffuse through the membrane and treat the infected cells from within. When the electrical field is withdrawn, the pores seal, and allows the drug agent within the cell to further treat the cell for a prolonged period of time.

An example of a system for treating infected tissue in accordance with one or more aspects of the present disclosure includes an applicator device. The applicator device incudes an electrode pair spaced apart and operable to be positioned against the infected tissue. An electrical power supply is operable to generate a pulsed electric field across the electrode pair, resulting in the application of a pulsed electric field through the infected tissue. The pulsed electric field has a field strength within a range that when the pulsed electric field is applied, pores are formed in membranes of cells of the infected tissue.

Another example of a system for treating infected tissue in accordance with one or more aspects of the present disclosure includes an applicator device. The applicator device includes an electrode pair spaced apart and operable to be positioned against the infected tissue. An electrical power supply is operable to generate an electric field having a field strength of 40 kV/cm or less across the electrode pair to apply a pulsed electric field being comprised of a plurality of electric field pulses having a time duration of about 50 to 900 nanoseconds through the infected tissue. The pulsed electric field has a field strength within a range that when the pulsed electric field is applied, pores are formed in membranes of cells of the infected tissue. An antimicrobial agent crosses or integrates into the membrane of the cells when the pulsed electric field is applied.

An example of a method of treating infected tissue in accordance with one or more aspects of the present disclosure includes positioning an electrode pair against the infected tissue. A pulsed electric field generated from a power supply is applied across the electrode pair through the infected tissue. The pulsed electric field has a field strength within a range that when the pulsed electric field is applied, pores are formed in membranes of cells of the infected tissue. An antimicrobial agent is applied into or onto the infected tissue when the pulsed electric field is applied, and allows the antimicrobial agent to cross or integrate into the membrane of the cells when the pulsed electric field is applied.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits and advantages described herein.

### BRIEF DESCRIPTION OF DRAWINGS

The disclosure will be more fully understood from the following detailed description taken in conjunction with the accompanying drawings, in which:

FIG. 1 depicts an example of a schematic block diagram of a system for treating infected tissue, according to aspects described herein;

FIG. 2 depicts an example of a plurality of wands as applicator devices of the system of FIG. 1, according to aspects described herein;

FIG. 3 depicts an example of a wand as an applicator device of the system of FIG. 1 being applied to a wound of a patient, according to aspects described herein;

FIG. 4 depicts an example of an electrical schematic of a plurality of electrode pairs disposed on a substrate as an applicator device, wherein the substrate is a flexible printed circuit board, according to aspects described herein;

FIG. 5 depicts an example of a bandage as an applicator device having the flexible printed circuit board and plurality of electrode pairs of FIG. 5 disposed thereon, according to aspects described herein;

FIG. 6 depicts an example of a screw as an applicator device, the screw having threads that includes the electrode pair of the system of FIG. 1 as a pair of wires wrapped around the screw, according to aspects described herein;

FIG. 7 depicts an enlarged view of the screw of FIG. 6 within the circle A of FIG. 6, according to aspects described herein;

FIG. 8 depicts an example of a complex shaped implant as an applicator device, the implant having a plurality of electrode pairs of the system of FIG. 1 disposed thereon, according to aspects described herein;

FIG. 9 depicts an example of a hip implant as an applicator device, the hip implant being disposed within a bone, the implant including a plurality of electrode pairs of the system of FIG. 1 disposed thereon, according to aspects described herein;

FIG. 10A depicts an example of an applicator device, which includes a hollow pin in fluid communication with a syringe, according to aspects described herein;

FIG. 10B depicts an enlarged view of the pin of FIG. 10A within the circle A of FIG. 10B, according to aspects described herein;

FIG. 11 depicts an example of an applicator device, which includes a negative pressure wound therapy device, according to aspects described herein;

FIG. 12 depicts an example of a bar chart of logarithmic reduction of S. epidermidis bacteria, according to aspects described herein;

FIG. 13 depicts an example of a bar chart of logarithmic reduction of P. aeruginosa bacteria, according to aspects described herein;

FIG. 14 depicts an example of a bar chart of logarithmic reduction of S. aureus bacteria, according to aspects described herein;

FIG. 15 depicts an example of a bar chart of logarithmic reduction of E. coli bacteria, according to aspects described herein;

FIG. 16 depicts an example of a bar chart of logarithmic reduction of P. aeruginosa biofilm, according to aspects described herein;

FIG. 17 depicts an example of a bar chart of logarithmic reduction of S. aureus biofilm, according to aspects described herein;

FIG. 18 depicts a flow diagram of a method of treating infected tissue, according to aspects described herein; and

FIG. 19A depicts a side view of a double lead combined device as an applicator device, according to aspects described herein;

FIG. 19B depicts an exploded side view of the double lead combined device of FIG. 19A, according to aspects described herein;

FIG. 19C depicts a side view of the double lead combined device of FIG. 19A, according to aspects described herein;

FIG. 20 depicts an exploded side view of a negative pressure wound therapy device (NPWT device), according to aspects described herein;

FIG. 21 depicts a hip stem as an applicator device, according to aspects described herein; and

FIG. 22 depicts an implant plate stack concept as an applicator device, according to aspects described herein.

### DETAILED DESCRIPTION OF THE INVENTION

Certain examples will now be described to provide an overall understanding of the principles of the structure, function, manufacture, and use of the methods, systems, and devices disclosed herein. One or more examples are illustrated in the accompanying drawings. Those skilled in the art will understand that the methods, systems, and devices specifically described herein and illustrated in the accompanying drawings are non-limiting examples and that the scope of the present disclosure is defined solely by the claims. The features illustrated or described in connection with one example maybe combined with the features of other examples. Such modifications and variations are intended to be included within the scope of the present disclosure.

The terms “significantly”, “substantially”, “approximately”, “about”, “relatively,” or other such similar terms that may be used throughout this disclosure, including the claims, are used to describe and account for small fluctuations, such as due to variations in processing from a reference or parameter. Such small fluctuations include a zero fluctuation from the reference or parameter as well. For example, they can refer to less than or equal to +10%, such as less than or equal to +5%, such as less than or equal to +2%, such as less than or equal to +1%, such as less than or equal to +0.5%, such as less than or equal to +0.2%, such as less than or equal to +0.1%, such as less than or equal to +0.05%.

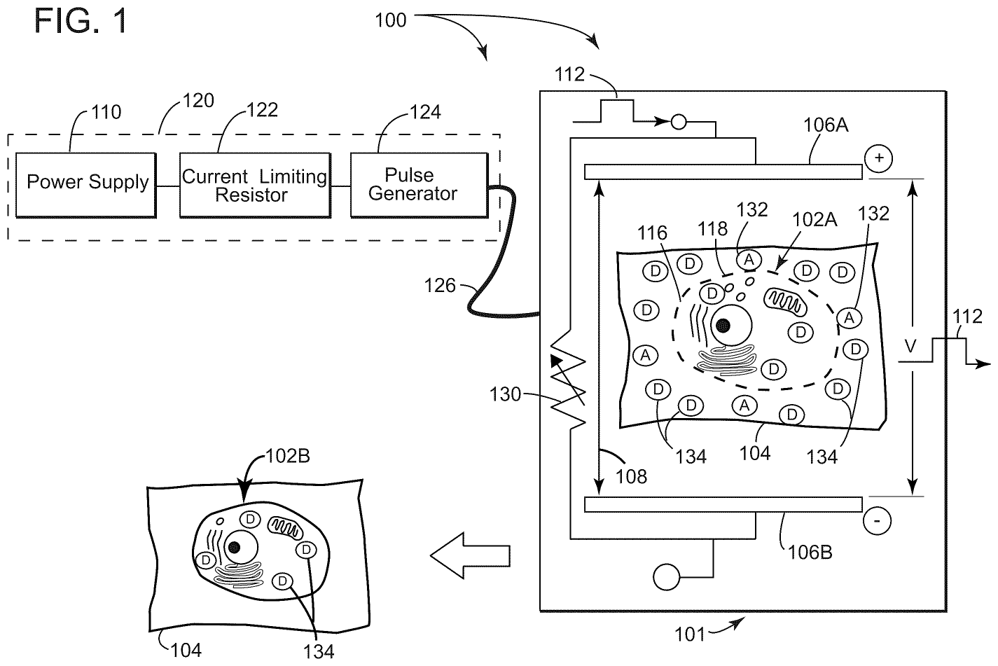

Referring to FIG. 1, an example is depicted of a schematic block diagram of a system for treating infected tissue 100, according to aspects described herein. The system 100 includes an applicator device 101, which includes an electrode pair 106A, 106B spaced apart a certain distance 108 and operable to be positioned against infected tissue 104 having cells 102A therein. The electrodes 106A, 106B may be composed of, for example, steel, titanium, nickel, tin or other electrically conductive material. The tissue 104 may be infected by, for example, bacteria. The electrode pair 106A, 106B may be, for example, spaced apart a distance 108 within a range of about 0.05 millimeters (mm) to 5 mm, within a range of 0.1 mm to 5 mm or within a range of 0.1 mm to 1.0 mm.

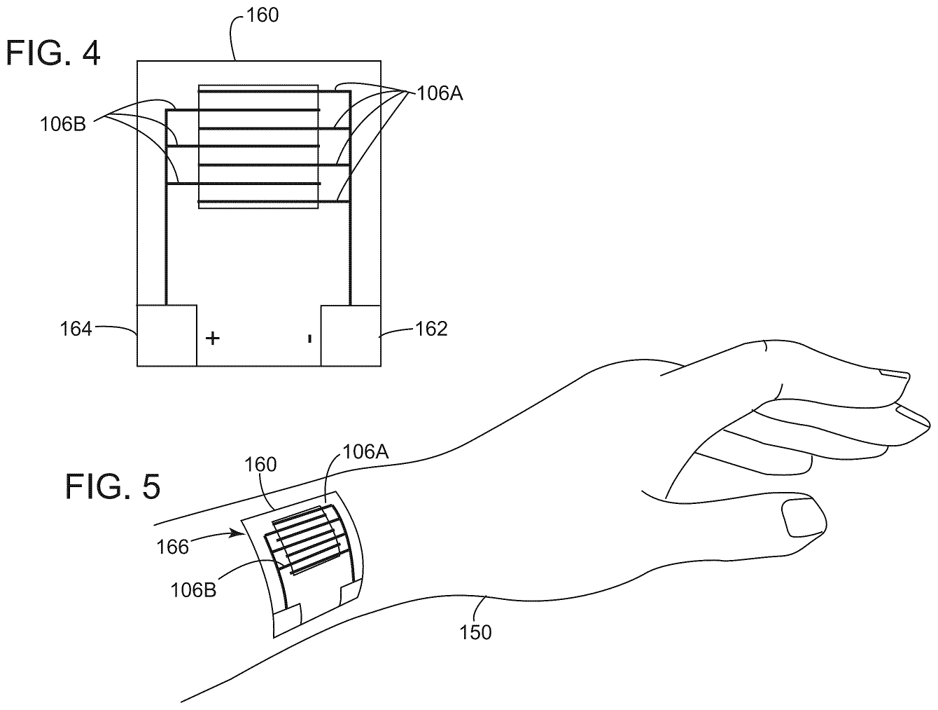

The applicator device 101 and the electrode pair 106A, 106B may be any appropriate configuration and structure, depending on the operating conditions and parameters required. For example, without limitation, the applicator device 101 may be a bandage 166 and the electrode pair 106A, 106B may be a single electrode pair or include a plurality of electrode pairs disposed on the bandage (see FIGS. 4 and 5). More specifically, the electrode pair 106A, 106B may include a plurality of parallel electrode pairs 106A, 106B disposed on a substate of the bandage (such as, for example, a flexible printed circuit board), wherein the electrode pairs 106A, 106B extend longitudinally across the substrate. The spacing 108 between each electrode 106A, 106B of the plurality of electrode pairs may be substantially equal to a width of each electrode of the plurality of electrode pairs. For example, the spacing 108 between the electrodes 106A, 106B and the widths of the electrodes 106A, 106B may be 100 microns, 50 microns or less. Alternatively, the spacing 108 between the electrodes 106A, 106B may differ from that of the width of the electrodes.

An electrical power supply 110 is configured to apply a pulsed electric field 112 across the electrode pair 106A, 106B to generate the pulsed electric field 112 through the infected cells 202A. The pulsed electric field 112 has a field strength within a range that when the pulsed electric field 112 is applied, pores 116 are formed in membranes 118 of the cells 102A of the infected tissue 104. Additionally, when the pulsed electric field 112 is withdrawn, the reversible pores 216 may seal (as exemplified by cell 202B). The field strength may be, for example, in the range of 40 kilovolts per centimeter (kV/cm) or less, 30 kV/cm or less, 20 kV/cm or less, 19 kV/cm or less, or 18 kV/cm or less.

Note, for purposes herein, the infected cells 102A and 102B are the same cell, but in two different states. Cell 102A is the cell wherein the electric field 108 is being applied and pores 116 are formed in the membrane 118 of the cell 102A. Cell 102B is the same cell, wherein the electric field 108 has been withdrawn and the pores 116 have closed or sealed.

Though a majority of pores 116 may seal when the pulsed electric field 112 is withdrawn (as exemplified by cell 102B), a minority of pores 116 on the cells 102A may not. For example, 60 percent (%), 70%, 80%, 90% or more of the pores 116 on the cells 102B may close or seal, but 100% may not seal.

The power supply 110 of the system 100 may be disposed in a system housing 120. The system housing 120 may be any appropriate configuration or structure, depending on the operating conditions and parameters required. For example, the system housing 120 may be a cabinet on wheels to be rolled around in a clinical setting. Also, by way of example, the system housing 120 may be a portable case that can be hand carried to remote locations.

The power supply 110 may conduct current through a current limiting resistor 122, which functions to reduce the magnitude of the pulsed electric field 112 to the cells 102A. The current limiting resistor 122 may be electrically connected to a pulse generator 124, wherein the pulsed electric field 112 is formed. The pulsed electric field may be composed of a plurality of electric field pulses 112 having a certain time duration. The duration of time of each pulse 112 may be, for example, about between 10 to 900 nanosecond (ns), between 50 to 900 ns, between 50 to 500 ns or between 50 to 300 ns.

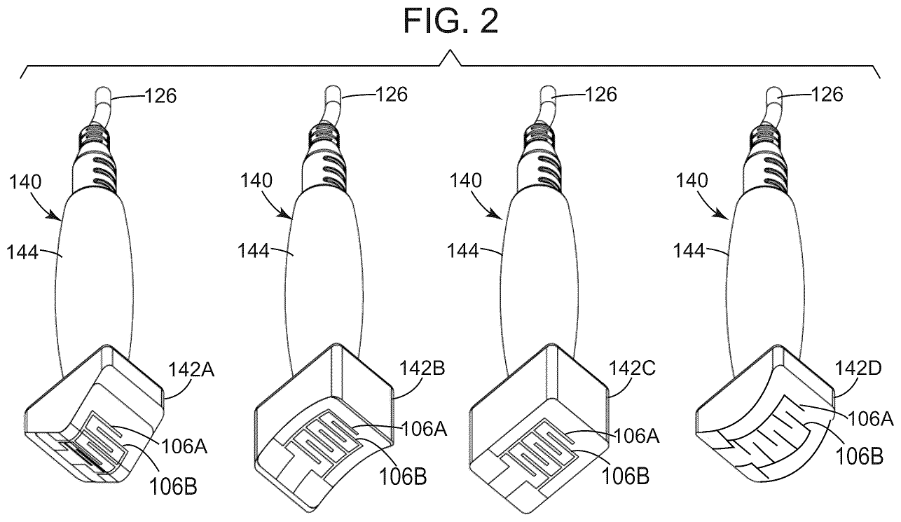

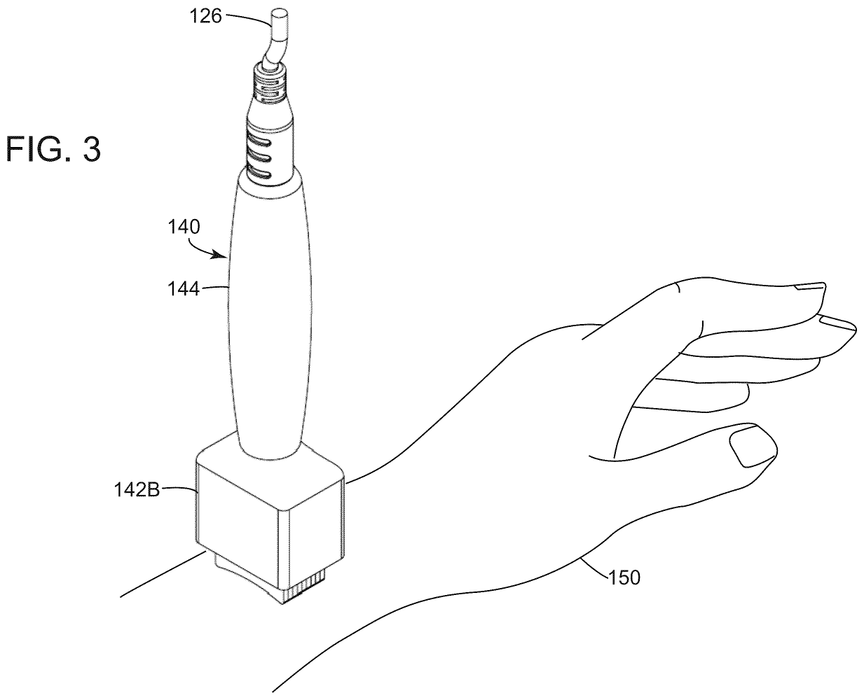

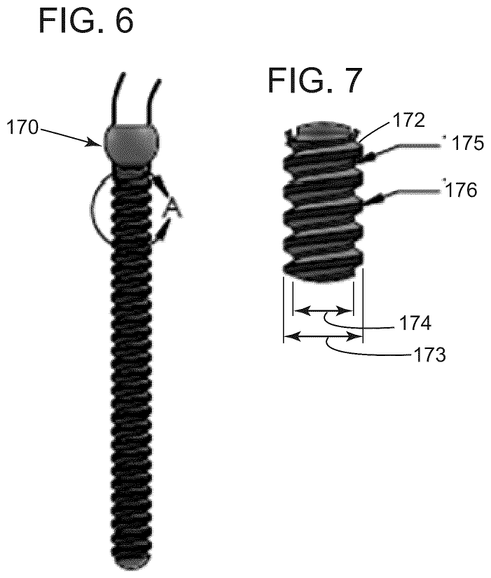

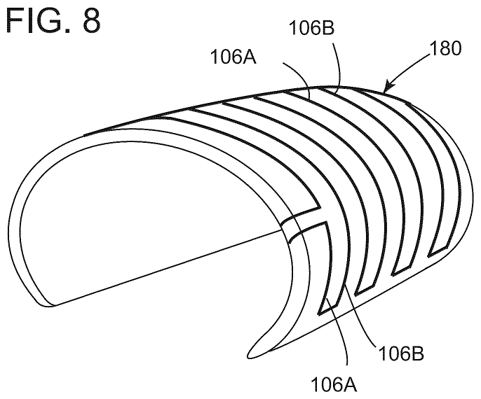

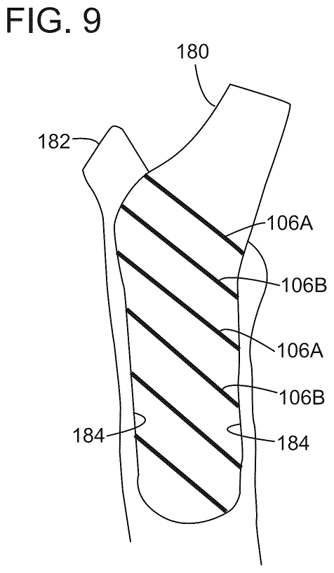

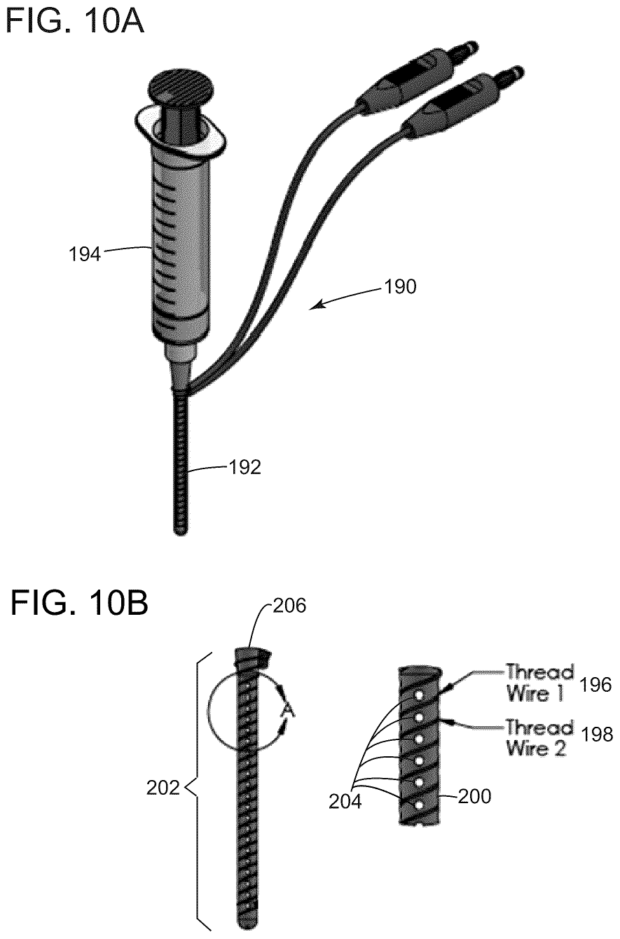

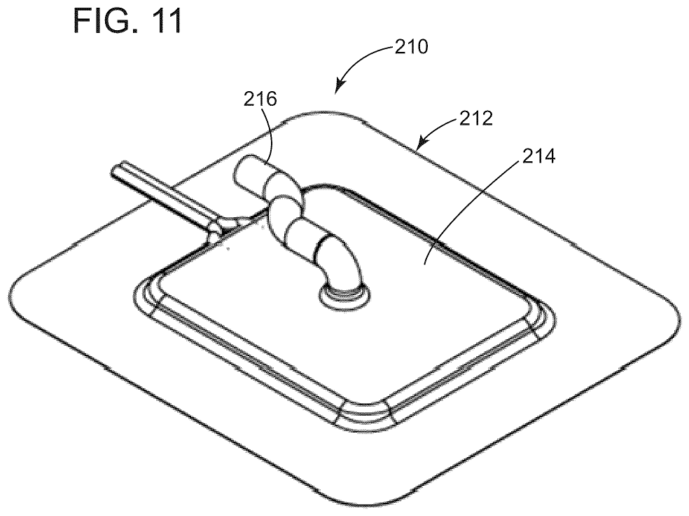

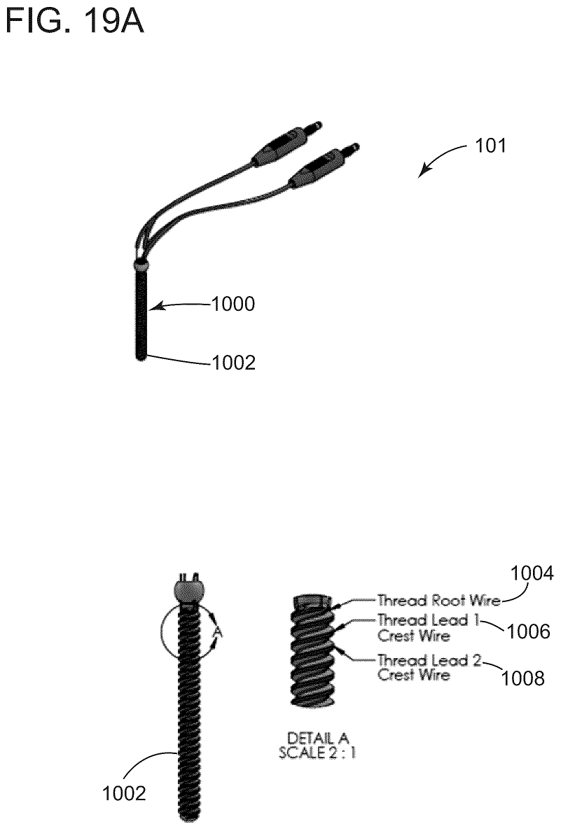

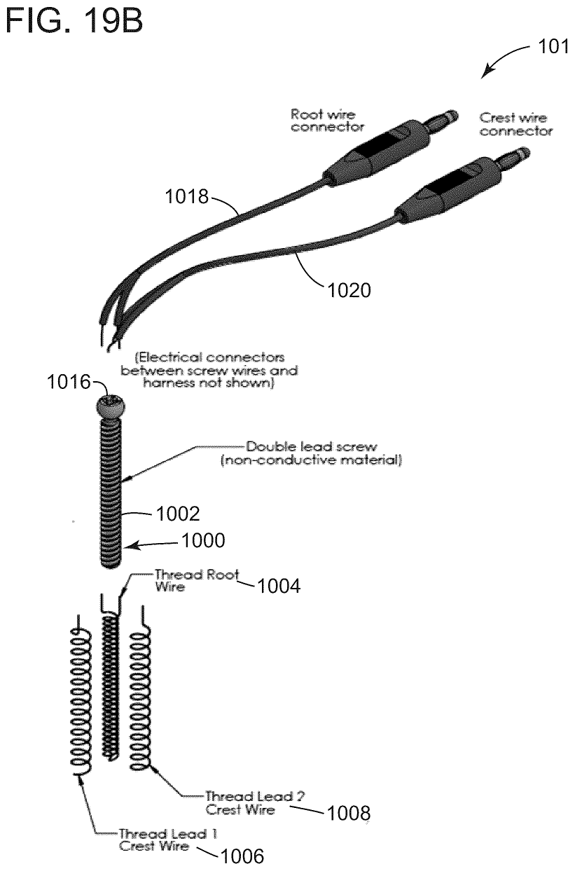

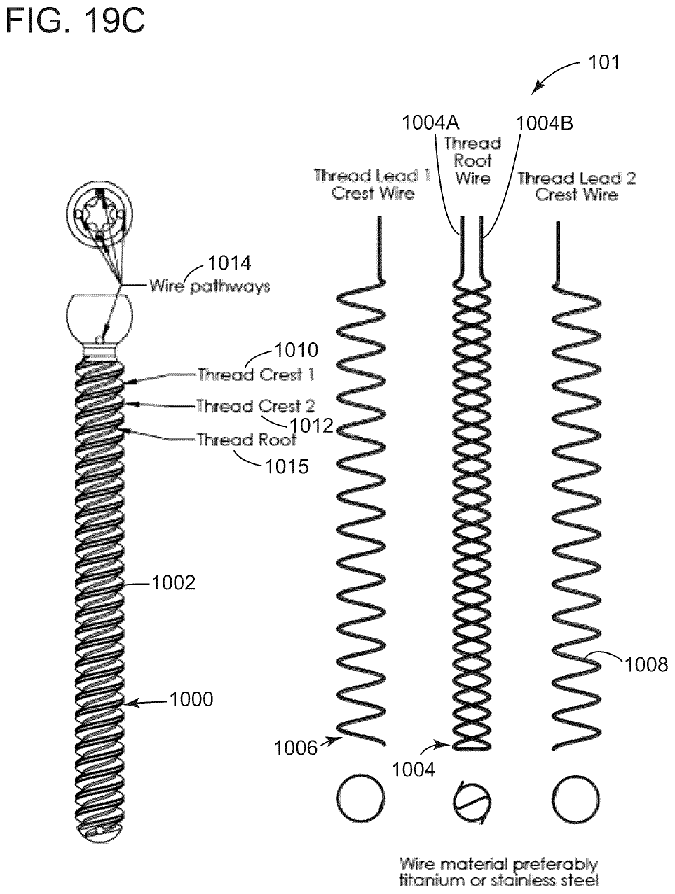

From the pulse generator 124, the pulsed electric field 112 may be conducted through a cable 126 to the applicator device 101, which is used to position the electrodes 106A, 106B proximate to the tissue 104 to be treated. The applicator device 101 may be any appropriate configuration or structure, depending on the operating conditions and parameters required. For example, without limitation, the applicator device 101 may include a hand-held wand (see FIGS. 2 and 3), a bandage (see FIGS. 4 and 4), a threaded screw (see FIGS. 6 and 7), a surgical implant with a complex geometry (such as a hip implant) (see FIGS. 8 and 9), a pin (see FIG. 10) or a negative pressure wound therapy device (NPWT device) (see FIG. 11). Additionally, and without limitation, many other embodiments of the applicator device 101 are described herein in greater detail with reference to FIGS. 19-22.

Applicator devices 101, such as a hand-held wand can apply the pulsed electric field 112 to the skin of a patient. Applicator devices 101, such as printed circuit boards allow the pulsed electric field 112 to be applied in a bandage and negative pressure wound therapy devices. Orthopedic applicator devices 101, such as external fixator pins, trauma plates, hips and knees are also possible. Temporary duration applicator devices 101, such as catheters, picc lines and vascular ports are also possible examples of methods and devices for applying a pulsed electric field. 3D printing of metals combined with resin casting, injection molding and compression molding enables the creation of more rigid applicator device 101 structures such as screws, bone probes, joint replacement hardware and other orthopedic and soft tissue integrating devices.

Applicator devices 101 may be used to treat skin infections. This would include burns, surgical site infections, general abrasions, diabetic ulcers, jock itch (fungal infections) and possibly cellulitis. The application of the pulsed electric field 112 is very short duration with a wand or bandage and is then removed. Advantageously, there is the high value in stopping/inhibiting infections with the low risk application from a regulatory perspective.

Application devices 101, such as bandages, negative pressure wound therapy, catheters, vascular access ports etc, may also be used for transient time periods. It is contemplated that treatment modes can be single or staged with longer duration implants, such as surgical plates, external fixator pins and permanent implants such as joint replacement devices may also be used.

Additionally, the system 100 and applicator devices 101 may be used to provide the following benefits, features and/or treatments:

A combination of pulsed electric fields with antibiotics to enhance microbial kill-off.
1) Pulsed electric fields to encourage the proliferation and differentiation of stem cells.
2) A time gap effect on delivery of pulsed electric fields (PEFs) to disrupt bacteria.
3) A combination of PEFs with systemic to local clinical to high dose antibiotics.
4) A combination of PEFs with multiple antibiotics with differing modes of action.
5) A combination of PEFs with a cooling pad to prevent thermal damage to tissue and limit bacterial proliferation. Pulsing at body temperature followed by cooling should cause the bacterial membranes to stiffen allowing pores to remain open longer, doing more damage to the cells.
6) A combination of PEFs with an antibiotic scaffold or injectable material to long lasting local antibiotics.
7) Electrodes loaded with antibiotics to be released when placed in or on the body.
8) Algorithm development for combining multiple PEF durations to disable a broad spectrum of microbes.
9) Algorithm development for specific bacterial species based on size and presence of biofilm.
10) Algorithm development to target specific bacterial resistance mechanisms, including:
   a. Frequency of delivery.
   b. Number of pulses needed to saturate for a given voltage and frequency.
   c. Detection of physical or enzymatic activity.
11) Electrode array combined with bandage and antibiotics, which may further provide:
   a. Single or multiple treatments.
   b. High localized antibiotic treatment to defeat resistant pathogens.
   c. Capability to work with oral and IV antibiotics.
   d. Regenerative pulsing to improve skin/tissue healing.
   e. Capability to be trimmed with scissors or scalpel to a specific size.
   f. Can come in a range of basic sizes.
   g. Bandage may have adhesive backing.
   h. Adhesive backing may cover a portion or all of the electrode array.
   i. Electrodes can be parallel lines with varying geometries.
   j. Electrodes can be point electrodes spaced evenly apart.
   k. Electrodes may be individually addressable.
12) Wand electrode to decontaminate skin wounds, burns, ulcers, bed sores, cellulitis, necrotizing fasciitis, surgical site infections, severe acne, trauma wounds, etc.
13) Flexible electrode for skin or surgical site decontamination.
14) Electrode width and spacing between electrodes may be varied to increase biofilm kill-off.
   Applies to implants, screws, bandages, wands, catheters, PICC lines, balloons, NPWT, stents, shunts.
   Using narrower electrodes creates a more consistent electric field across the surface.
   Electrodes or electrical traces on PCBs as narrow as 10 microns up to 5,000 microns (5 millimeters).
   Spacing between electrodes from 10 microns to 10,000 microns (10 millimeters).
   Narrower electrode spacing allows use of higher electric field strength with lower penetration into the host tissue.
   Targets colonizing bacteria and biofilms on the surface of a device/implant.
   Electrode composition may include gold, silver, copper, aluminum, titanium, stainless steel, Nitinol.
   Nitinol is a shape memory alloy that can unfold into a prespecified shape when deployed.
      i. Similar to stents but with pulseable circuits.
      ii. Can be embodied as a stent or shunt.
15) Electrode bandage combined with bioactive materials.
   Bioactive material can encapsulate or be coated with the antibiotics
   Hydrogels to help maintain moisture levels and allow healing
   Gand collagen sponges
   Natural and synthetic hemostats
   Extracellular matrices to enable healing and regeneration of lost tissue
16) Vacuum drainage bandage with pulsing electrodes.
   Combine with antibiotic wash to enhance local pathogenic microbe kill-off.
   First version helps eliminate bacteria in the absorbent pad.
   Second version helps eliminate bacteria in the underlying wound.
   Absorbent pad can be loaded with antibiotics.
   Single or double access ports.
17) Bone probe electrode to decontaminate interior of bone tissue during implant surgery or revision.
18) Spine interbody implant device.
19) Bone screw or pin which includes two electrodes that are positioned on an outer surface of the body of the screw or pin (see FIGS. 6A and 9). Optionally, a central electrode (not shown) may be positioned within a central channel of the screw or pin, wherein the central electrode is insulated from the outer two electrodes and exposed at a tip of the screw or pin. Additionally, the central electrode may be electrically connected to one of the outer two electrodes.
20) Hip stem spacer (temporary implant to deal with infection).
21) Laparoscopic electrode
22) Urinary catheters
   Electrodes run the length of the catheter on the outside.
   Lubrication gel for insertion could contain antibiotics.
   Antibiotics can be incorporated into the hydrogel layer on exterior of device.
   Antibiotics could be directly deposited in the bladder through the catheter for residual drug exposure and cleared through the urethra.
   Treatment of multidrug resistant urinary tract infections.
23) Peripherally Inserted Central Catheter (PICC) line.
   Electrodes line the internal length of the catheter on the inside and a short distance of the external area of the tip (where biofilms typically occur).
   Transdermal skin passage.
24) Transdermal devices.
   Ostomy ports
   Feeding tubes
   Vascular ports
25) Balloon devices
   A.) Anatomical targets
      Arterial/venous stent or vessel wall
      Bladder
      Cysts/abscesses
      Vagina
      Sinus and nasal cavities
      Ear canal
      Oral cavity
      Gastrointestinal tract
   B.) Parallel lines of electrodes paired together maintain distance as balloon is expanded.
   C.) Balloons can be filled with air in non-critical sites or saline in critical sites such as blood vessels.
26.) Electrodes can be individually electrically addressable
   Helps to better target sites of infection
   Limits off-target host tissue damage
   A mask can be further added to cover and protect healthy tissue.

The system 100 may also include a bridging resistor 130 electrically connected in parallel with the electrode pair 106A, 106B to the power supply 110. The bridging resistor may be used to substantially match an impedance of the pulse generator 124. The bridging resistor 130 may be an adjustable bridging resistor that is operable to be adjusted to match the impedance of the pulse generator 124.

The bridging resistor 130 helps to ensure that the electric field pulses 112 are clean and normal with minimal reflections. The clean pulses 112 help enable the electrodes 106A, 106B to deliver the same pulse wave to the target tissue 104, regardless of specific conditions in the host body of the target tissue 104.

The system 100 may also include an antimicrobial agent 132 that is operable to cross or integrate into the membrane 118 of the cells 102A when the pulsed electric field 112 is applied. Advantageously, the synergistic combination of the antimicrobial agent 132 being applied simultaneously with the pulsed electric field 112, enables a drug agent 134, that is operable to treat the cells 102A, to more efficiently cross or integrate into the membrane of the cells 102A. Also, the antimicrobial agent 132 may be applied in dilute concentrations. For example, the concentration of the antimicrobial agent 132 may be between 0.001 percent and 2.0 percent.

The drug agent 134, as used herein includes, for example, at least one of an antibiotic, an antiseptic or an antifungal agent that is operable to cross or integrate into the membrane 118 of the cells 102A when the pulsed electric field 112 is applied. For example, the drug agent 134 may be Tobramycin, Rifampicin, Clindamycin, Tetracyclines, Carbapenems, Cephalosporins, glycopeptides, Lincosamides, Lipopeptides, Macrolides, Oxazolidinones, Penicillins, Quinolones, Fluoroquinolones, Tigecycline, Mupirocin, Chloramphenicol and others. As will be described and illustrated in greater detail herein with regards to FIGS. 11-16, the microorganism inactivation rate of the at least one of an antibiotic, an antiseptic or an antifungal agent 134 is greater when the pulsed electric field 112 and antimicrobial agent 132 are applied simultaneously than when the pulsed electric field 112 and antimicrobial agent 132 are applied separately.

Once the pulsed electric field 112 is removed, the pores 216 may heal or reseal (as exemplified in cell 102B). Accordingly, the drug agent 134 gets trapped within the interior of the cell 202B where it can further treat the cell for a prolonged period of time.

The antimicrobial agent 132 may be applied in relatively small concentrations to avoid any potential side effects. For example, the concentration of the antimicrobial agent 132 may be between 0.005 percent and 0.02 percent or between 0.001 percent and 2.0 percent. As will be explained in greater detail herein, such concentrations of Chlorhexidine in combination with the pulsed electric field 112 and the antibiotic agent Tobramycin have resulted in significant reductions of various bacteria, including various biofilm coated bacteria (see FIGS. 11-16).

Chlorhexidine 132 is an antimicrobial agent 132 that inserts across or integrates into the cell membrane 118. The Chlorhexidine molecule 132 and other members of the biguanide family cause cell stress by allowing leakage of ions across the membrane 118. Bacterial cells 102A use the cell membrane 118 to create potential energy in the form of increased osmolarity inside the cell 102A and imbalances in ion concentrations inside and outside the cell 102A This is akin to a hydroelectric dam with a reservoir of water to power the creation of electricity. In the case of the bacteria, the potential energy is used to pump undesired molecules out of the bacterial cell via efflux pumps and power ATP or GTP dependent enzymes by creating adenosine triphosphate (ATP) or guanosine triphosphate (GTP), these are antibiotic resistance mechanisms.

Chlorhexidine 132 and other biguanides cross or integrate into the membrane 118 of the cell 102A and help drain the reservoir. Without the store of potential energy, bacterial cells 102A are dependent upon readily available sources of nutrients to make ATP to power the bacterial cell. If nutrients are scarce, such as in a biofilm bacteria, the bacterial cells can be overwhelmed more quickly by environmental factors and the drug agent 134.

Chlorhexidine 132 can also increase the effectiveness of other antibiotics 134 by increasing the ability of antibiotics 134 to diffuse through the membrane 118 via the ion leakage across the membrane 118.

Concentrations of the drug agent 134 in the cells 102A can remain higher for longer periods of time as the cells 102A have reduced capacity to pump the drug agent 134 out of the cells 102A through a reduction in stored potential energy enabled by forming pores with pulsed electric fields.

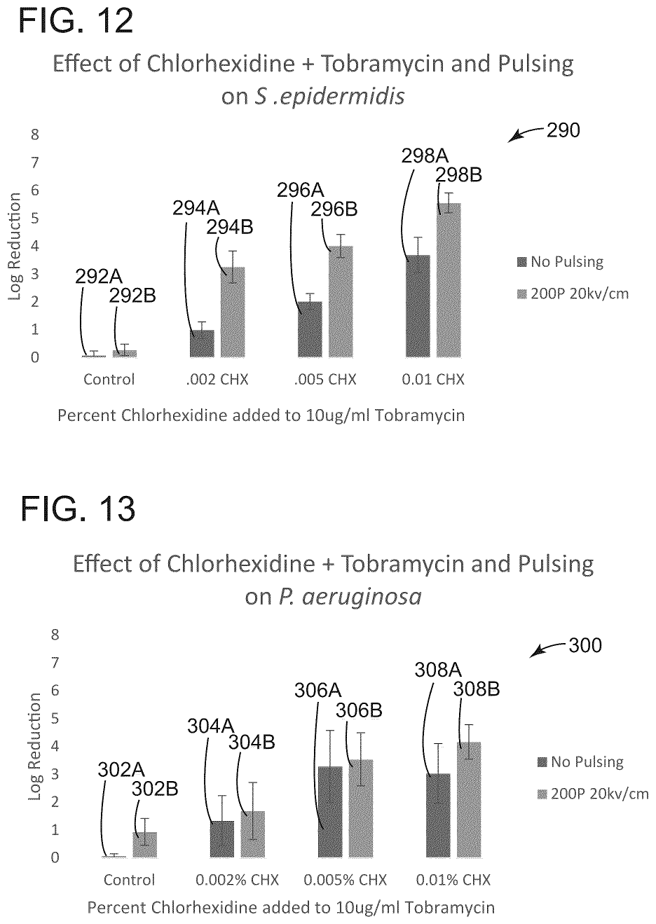

Chlorhexidine is potentially toxic to host tissue cells 104 when administered in high enough concentrations. Chlorhexidine has been used as a topical scrub in a concentration between 1% and 3%. However, using a concentration of Chlorhexidine between 0.005% and 0.01% in combination with tobramycin (i.e., drug agent 134) achieves a substantial kill-off without the potential toxic side effects that inhibit tissue healing. Advantageously, when such reduced Chlorhexidine concentrations is combined with a pulsed electric field 112 there is an increased synergy in kill-off that allows for multiple log to the base 10 kill-off of several planktonic bacterial species in 5 to 15 minutes of the drug agent 134 exposure, beyond what the drug can achieve alone (see FIGS. 12-16).

When applied such Chlorhexidine concentrations alone (i.e., without a pulsed electric field 112) to bacteria coated with biofilms, the drug agents 134 have less of an effect. Applying a pulsed electric field 112 alone (i.e., without Chlorhexidine or other antimicrobials) to bacterial biofilms has a greater effect on incapacitating the bacteria than when the bacteria are in the planktonic state. Advantageously, combining a pulsed electric field 112 with the chlorhexidine and tobramycin (i.e., drug agent 134) combination a log to the base 10 kill-off can be achieved in 15 minutes of drug exposure with a single 200 pulse treatment (see FIG. 17). Increasing to 1000 pulses may result in a 2-log kill-off of mature 72 hour Pseudomonas aeruginosa biofilms.

Besides Chlorhexidine, there are several other antimicrobial agents 132 that may be used. For example, polyaminopropyl biguanide (PAPB), polihexanide, and alexidine. Additionally, such antimicrobial agents are defined and described in PCT Published Patent Application No.: WO 2022/132628, filed on Dec. 13, 2021, the contents of which are incorporated herein in their entirety.

Referring to FIG. 2 an example is depicted of a wand 140 used as an applicator device 101, wherein the wand 140 has a plurality of different wand heads 142A, 142B, 142C, 142D according to aspects described herein. Each of the wands 140 have a handle 144 attached to a head 142A, 142B, 142C, 142D. On the distal end of each head 142A, 142B, 142C, 142D a plurality of electrode pairs 106A, 106B are disposed. The plurality of parallel electrode pairs 106A, 106B extend longitudinally across each head. The spacing between each electrode 106A, 106B of the plurality of electrode pairs is substantially equal distance apart. The wands 140, and therefore the electrodes 106A, 106B within the wands 140, are electrically connected to the power supply 110 of the system 100 via cable 126, which is connected to the rear end of the wands 140.

Each of the wands 140 are operable to be grasped by the handle 140 by a user 146 (see FIG. 3) and the distal end of the head 142A, 142B, 142C, 142D of each wand 140 is operable to be pressed by the user 146 against the infected tissue 104. The heads 142A, 142B, 142C, 142D of each of the wands 140 have different shaped distal ends to conform to the different geometries of a patient's body. More specifically, head 142A has a triangular distal end, head 142B has a concave distal end, head 142C has a flat distal end and head 142D has a convex distal end.

Referring to FIG. 3, an example is depicted of a wand 140 of the system 100 being applied by a user (not shown) to a wound (not shown) of a patient 150, according to aspects described herein. The wound can be due to a burn, physical trauma or an infection or the like. The user (not shown) can be a doctor, an emergency medical technician, a nurse or the like, that grasps the handle 144 of the wand 140 and applies the distal end of the head 142B against the patient 150. The wand 140 may be pressed against any portion of the body of the patient 150, such as, for example, a leg, arm, foot or the like. The head 142B of the wand 140 has a concave distal end. However, any of the heads 142A, 142B, 142C, 142D may be used depending on the shape of the portion of the patient's body to which the wand 140 conforms.

Referring to FIG. 4, an example is depicted of an electrical schematic of the applicator device 128 in the form of a plurality of electrode pairs 106A, 106B disposed on a substrate 160, in accordance with aspects described herein. The electrodes 106A, 106B extend longitudinally across the substrate 160. The substrate 160 may be any appropriate underlying material for the plurality of electrodes 106A, 106B to be disposed upon. The substrate 160 is operable to form a bandage 166 for placement over the infected tissue 104. In this case, the substrate 160 is a flexible printed circuit board 160. The printed circuit board 160 also includes a pair of terminals 162, 164 that are electrically connected to the electrodes 106A, 106B. The terminals may be electrically connected to the power supply 110 via cable 126.

Referring also to FIG. 5, an example is depicted as bandage 166 having the flexible printed circuit board 160 and plurality of electrode pairs 106A, 106B disposed thereon, according to aspects described herein. Placing the electrodes 106A, 106B on a flexible printed circuit board 160, enables a flexible bandage 166 to be constructed. The bandage 166 may include an adhesive layer (not shown) that can adhere to various parts of a patient's body, regardless of the shape of the parts.

Referring to FIGS. 6 and 7, an example is depicted of the applicator device 128 in the form of a screw fastener 170 (FIG. 6) configured to be threaded into the infected tissue 104, for example, human bone and an expanded view (FIG. 7) of the screw of FIG. 6 within the circle A of FIG. 6.

The screw fastener 170 has threads 172 that include a major diameter 173 and a minor diameter 174. A pair of electrodes include a first electrode 175 wrapped around the minor diameter 174 and a second electrode 176 wrapped around the major diameter 173. The first and second electrodes 175, 176 may be, any appropriate configuration required to meet application parameters. For example, the first and second electrodes may be wires, laser deposited electrodes or the like. The first and second electrodes 175, 176 may be composed of steel, titanium or the like. The screw fastener may be manufactured via 3D printing or the like. The screw fastener 170 may be composed of PEEK, PEKK, PMMA, Epoxy, polypropylene, polyethylene, thermoplastic, thermoset or other non-conductive materials. A steel or metal core may be used to increase the strength of the screw fastener 170. Additionally, the non-conductive material may be biodegradable to allow easy removal of the fastener wires or core material. Biodegradable material may be composed of polymers such as PGA, PLGA, PCL, and others or the material can be a biodegradable ceramic such as calcium phosphate, calcium sulphate, tricalcium phosphate, hydroxyapatite or other biodegradable ceramic or bioglass.

Referring to FIG. 8, an example is depicted of the applicator device 128 in the form of complex shaped implants 180, in accordance with aspects described herein. The implants are operable to be inserted into the infected tissue 104. One or more electrode pairs 106A, 106B are disposed on an outer surface of the implant 180. In this case, electrodes 106A, 106B are illustrated as a plurality of electrode pairs 106A, 106B that are disposed on a flexible printed circuit board that is disposed over an outer surface of the implants 180.

The tissue 104 may be human bone. The implants may be, for example, one of a plate, a knee implant, a shoulder implant, an elbow implant, an ankle implant, a spine interbody implant or a hip stem.

Referring to FIG. 9, an example is depicted of an implant 180 disposed within a hip bone 182 of a patient. The implant 180 as shown, is a hip stem and includes a plurality of electrode pairs 106A, 106B of the system 100 disposed thereon. The implant 180 may be inserted into a cavity 184 that was surgically bored into the bone 182 to treat adjacent infected tissue. When the infection has been treated by the system 100, the implant 180 may be removed and a permanent implant (not shown) may then be inserted. Alternatively, the implant 180 may be permanently inserted into the cavity 184. The permanent implant may also include antimicrobial properties and/or integrated circuitry for future treatments in situ.

Referring FIG. 10A, an example is depicted of an applicator device 190 (an embodiment of applicator device 101), which includes a hollow pin 192 in fluid communication with a syringe 194, according to aspects described herein. The pin 190 is operable to be inserted into the infected tissue 104. The applicator device 190 includes a first electrode 196 and a second electrode 198, which form a pair of electrodes. The first and second electrodes 196, 198 may be, any appropriate configuration required to meet application parameters. For example, the first and second electrodes may be wires, laser deposited electrodes or the like. The first and second electrodes 196, 198 are wrapped around an outside surface 200 of a shaft 202 of the pin 192. The first and second electrodes 196, 198 may be evenly spaced apart along the shaft 202 of the pin 192 or parallel to each other along the shaft of the pin.

The pin 192 may be hollow or solid. In the example illustrated in FIG. 10B, the pin 192 is hollow and has an inside surface, wherein the inside surface defines a central channel (not shown), The pin includes a plurality of evenly spaced through holes 204 disposed along the shaft 202 of the pin 192. The through holes 204 extend from the channel to the outside surface 200 of the pin 192.

Optionally, a central electrode (not shown) may be positioned within the central channel of the pin 192. The central electrode may be insulated from the first and second electrodes 196, 198 (i.e., the outer two electrodes) and exposed at a tip of the pin 192. Additionally, the central electrode may be electrically connected to one of the outer two electrodes.

The syringe 194 is operable to be connected to a distal end 206 of the pin 192 such that the syringe 194 is in fluid communication with the channel. The syringe 194 is operable to deliver fluids into the channel and out through the through holes 204. The fluids may include, for example, at least one of an antimicrobial agent, antibiotic agent, an antifungal agent or antiseptic agent.

Referring to FIG. 11, an example is depicted of an applicator device 210 (an embodiment of applicator device 101), which includes a negative pressure wound therapy device (NPWT device) 212, according to aspects described herein. The underlying structure of the applicator device 210 is similar to the bandage 166 as described herein in FIGS. 4 and 5. Additionally, the applicator device 210 includes the NPWT device 212 (also referred to herein as vacuum-assisted wound closure device), which includes a wound dressing system 214 that continuously or intermittently applies sub-atmospheric pressure (i.e., vacuum pressure) to the surface of a wound on a patient. The vacuum pressure is pulled through a vacuum line 216 that is connected to a vacuum pump (not shown). The pressure applied may be used to pull or pump fluid and/or tissue and/or cells from a patient's wound. The NPWT device 212 in accordance with the present disclosure may, in one embodiment, include an open-pore polyurethane ether foam sponge, a semi-occlusive adhesive cover, a fluid collection system, and a vacuum pump (not shown).

The applicator device 210 also includes a pair of electrodes 106A, 106B (not shown), which are pressed against the wound of the patient. The pair of electrodes 106A, 106B are disposed on the underside of the NPWT device 212. The pair of electrodes 106A, 106B may include a plurality of pairs of electrodes 106A, 106B.

Referring to FIG. 12, an example is depicted of a bar chart 290 of logarithmic reduction of Staphylococcus (S.) epidermidis bacteria, according to aspects described herein. The bar chart 290 shows the log to the base 10 reduction of the S. epidermidis bacteria after being subjected to various concentrations of Chlorhexidine with 10 micrograms per milliliter (μg/mL) Tobramycin antibiotic, both with and without 200 pulses of an electric field having a field strength of 20 kV/cm.

Bars 292A and 292B are the control group of no pulsing (292A) versus pulsing (292B), respectively. Bars 294A and 294B show the log reduction of a concentration of 0.002% Chlorhexidine with 10 μg/mL Tobramycin antibiotic, both without pulsing (294A) and with pulsing (294B). Bars 296A and 296B show the log reduction of a concentration of 0.005% Chlorhexidine with 10 μg/mL Tobramycin antibiotic, both without pulsing (296A) and with pulsing (296B). Bars 298A and 298B show the log reduction of a concentration of 0.01% Chlorhexidine with 10 μg/mL Tobramycin antibiotic, both without pulsing (298A) and with pulsing (298B).

In each case, the combination of the Chlorhexidine and Tobramycin antibiotic solution combined with a pulsed electric field showed a dramatic increase in the reduction of bacteria compared to the Chlorhexidine and Tobramycin antibiotic solution alone.

Referring to FIG. 13, an example is depicted on a bar chart 300 of logarithmic reduction of Pseudomonas (P.) aeruginosa bacteria, according to aspects described herein. The bar chart 300 shows the log to the base 10 reduction of the S. aureus bacteria after being subjected to various concentrations of Chlorhexidine with 10 μg/mL Tobramycin antibiotic, both with and without 200 pulses of an electric field having a field strength of 20 kV/cm.

Bars 302A and 302B are the control group of no pulsing (302A) versus pulsing (302B), respectively. Bars 304A and 304B show the log reduction of a concentration of 0.002% Chlorhexidine with 10 μg/mL Tobramycin antibiotic, both without pulsing (304A) and with pulsing (304B). Bars 306A and 306B show the log reduction of a concentration of 0.005% Chlorhexidine with 10 μg/mL Tobramycin antibiotic, both without pulsing (306A) and with pulsing (306B). Bars 308A and 308B show the log reduction of a concentration of 0.01% Chlorhexidine with 10 μg/mL Tobramycin, both without pulsing (308A) and with pulsing (308B).

In each case, the combination of the Chlorhexidine and Tobramycin antibiotic solution combined with a pulsed electric field showed a dramatic increase in the reduction of bacteria compared to the Chlorhexidine and Tobramycin antibiotic solution alone.

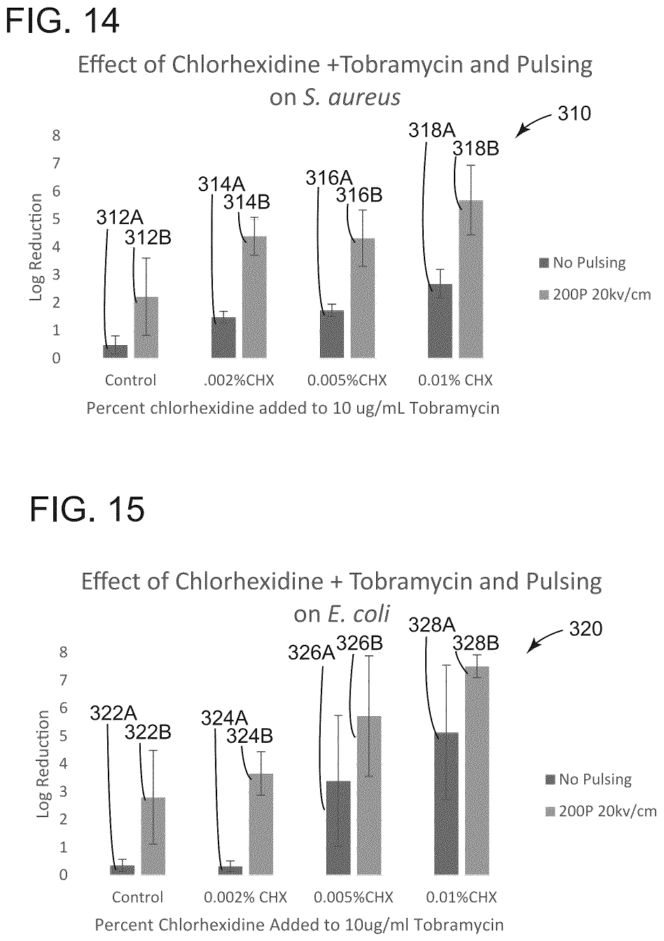

Referring to FIG. 14, an example is depicted of a bar chart 310 of logarithmic reduction of S. aureus bacteria, according to aspects described herein. The bar chart 310 shows the log to the base 10 reduction of the S. aureus bacteria after being subjected to various concentrations of Chlorhexidine with 10 μg/mL Tobramycin antibiotic, both with and without 200 pulses of an electric field having a field strength of 20 kV/cm.

Bars 312A and 312B are the control group of no pulsing (312A) versus pulsing (312B), respectively. Bars 314A and 314B show the log reduction of a concentration of 0.002% Chlorhexidine with 10 μg/mL Tobramycin antibiotic, both without pulsing (314A) and with pulsing (314B). Bars 316A and 316B show the log reduction of a concentration of 0.005% Chlorhexidine with 10 μg/mL Tobramycin antibiotic, both without pulsing (316A) and with pulsing (316B). Bars 318A and 318B show the log reduction of a concentration of 0.01% Chlorhexidine with 10 μg/mL Tobramycin antibiotic, both without pulsing (318A) and with pulsing (318B).

In each case, the combination of the Chlorhexidine and Tobramycin antibiotic solution combined with a pulsed electric field showed a dramatic increase in the reduction of bacteria compared to the Chlorhexidine and Tobramycin antibiotic solution alone.

Referring to FIG. 15, an example is depicted of a bar chart 320 of logarithmic reduction of Escherichia (E.) coli bacteria, according to aspects described herein. The bar chart 320 shows the log to the base 10 reduction of the E. coli bacteria after being subjected to various concentrations of Chlorhexidine with 10 μg/mL Tobramycin antibiotic, both with and without 200 pulses of an electric field having a field strength of 20 kV/cm.

Bars 322A and 322B are the control group of no pulsing (322A) versus pulsing (322B), respectively. Bars 324A and 324B show the log reduction of a concentration of 0.002% Chlorhexidine with 10 μg/mL Tobramycin antibiotic, both without pulsing (314A) and with pulsing (324B). Bars 326A and 326B show the log reduction of a concentration of 0.005% Chlorhexidine with 10 μg/mL Tobramycin antibiotic, both without pulsing (326A) and with pulsing (326B). Bars 328A and 328B show the log reduction of a concentration of 0.01% Chlorhexidine with 10 μg/mL Tobramycin antibiotic, both without pulsing (328A) and with pulsing (328B).

In each case, the combination of the Chlorhexidine and Tobramycin antibiotic solution combined with a pulsed electric field showed a dramatic increase in the reduction of bacteria compared to the Chlorhexidine and Tobramycin antibiotic solution alone.

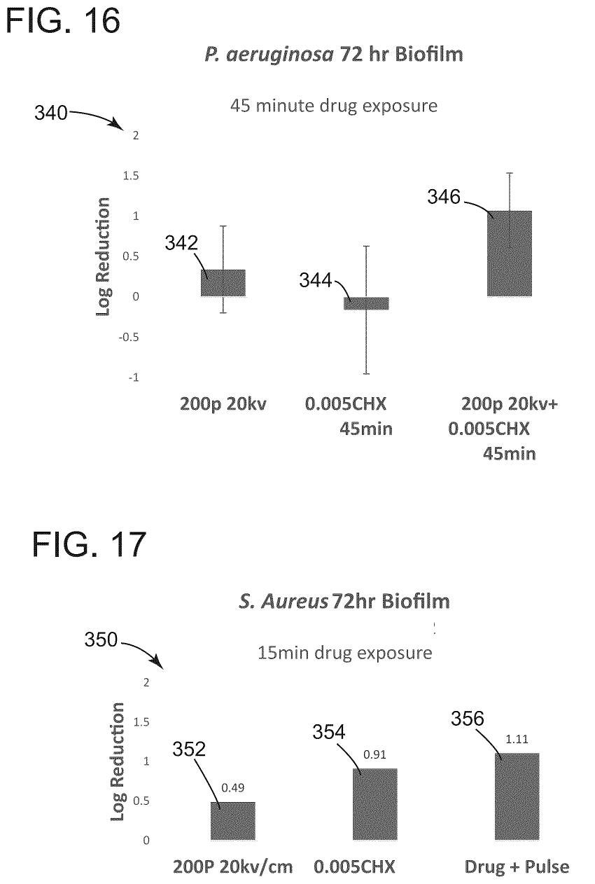

Referring to FIG. 16, an example is depicted of a bar chart 340 of logarithmic reduction of P. aeruginosa biofilm, wherein the biofilm has been allowed to grow over a 72 hour period, according to aspects described herein. The bar chart 340 shows the log to the base 10 reduction of the P. aeruginosa biofilm after being subjected to a treatment of: 1) a concentrations of 0.005% Chlorhexidine with 10 μg/mL Tobramycin antibiotic for a 45 minute period (i.e., the drug), or 2) 200 pulses of an electric field at a field strength of 20 kV/cm (i.e., the electric field), or 3) both the pulsed electric field and drug together.

Bar 342 shows the logarithmic reduction of the P. aeruginosa biofilm when treated with the pulsed electric field alone. Bar 344 shows the logarithmic reduction of the P. aeruginosa biofilm when treated with the drug alone. Bar 346 shows the logarithmic reduction of the P. aeruginosa biofilm when treated with both the pulsed electric field and drug.

Bar 346 (the application of the drug and pulsed electric field combined) shows a dramatic increase in the reduction of P. aeruginosa biofilm compared to bar 342 (the application of the pulsed electric field alone) and bar 344 (the application of the drug alone).

Referring to FIG. 17, an example is depicted of a bar chart 350 of logarithmic reduction of S. aureus biofilm, wherein the biofilm has been allowed to grow over a 72 hour period, according to aspects described herein. The bar chart 350 shows the log to the base 10 reduction of the S. aureus biofilm after being subjected to a treatment of: 1) a concentrations of 0.005% Chlorhexidine with 10 μg/mL Tobramycin antibiotic for a 15 minute period (i.e., the drug), or 2) 200 pulses of an electric field at a field strength of 20 kV/cm (i.e., the electric field), or 3) both the pulsed electric field and drug together.

Bar 352 shows the logarithmic reduction of the S. aureus biofilm when treated with the pulsed electric field alone. Bar 354 shows the logarithmic reduction of the S. aureus biofilm when treated with the drug alone. Bar 356 shows the logarithmic reduction of the S. aureus biofilm when treated with both the pulsed electric field and drug.

Bar 356 (the application of the drug and pulsed electric field combined) shows a dramatic increase in the reduction of S. aureus biofilm compared to bar 352 (the application of the pulsed electric field alone) and bar 354 (the application of the drug alone).

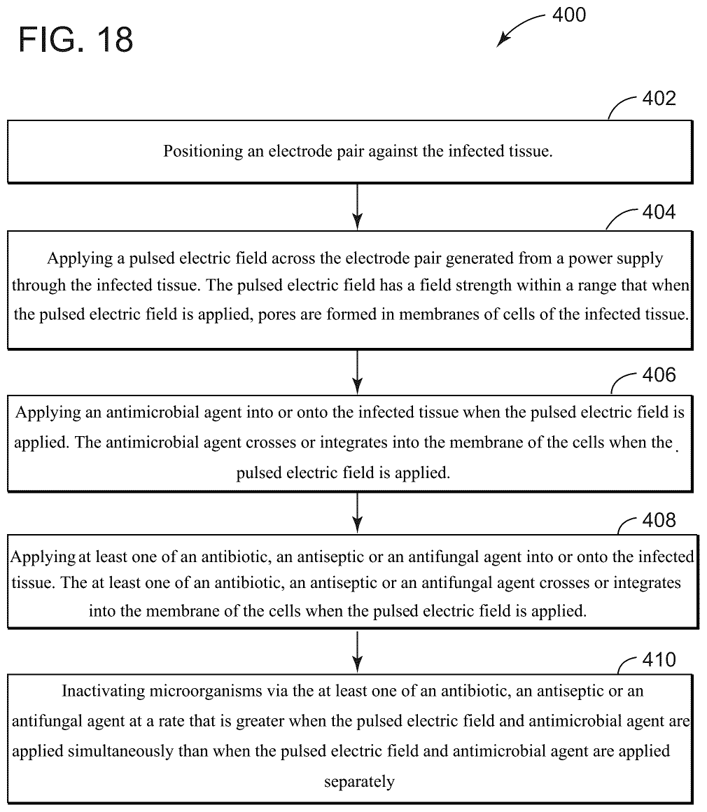

Referring to FIG. 18, an example of a flow diagram of a method 400 of treating infected tissue, in accordance with aspects described herein is shown. The method 400 follows a treatment process that may be followed using the system 100 on infected tissue 104. Though the following method steps are shown and described in a certain order, the method steps may be performed in any reasonable order, depending on the conditions and parameters required.

At 402 of the method 400, an electrode pair 106A, 106B is positioned against the infected tissue 104.

The electrodes 106A, 106B may be spaced apart a certain distance 108. The distance 108 may be within a range of 0.1 mm to 5 mm.

Additionally, the electrode pair 106A, 106B may be disposed within an applicator device 101. The applicator device 101 may be, for example, a wand, a pin, a screw, a bandage, a negative pressure wound therapy device or an implant.

At 404, a pulsed electric field 112, generated from a power supply 110, is applied across the electrode pair 106A, 106B and through the infected tissue 104. The pulsed electric field 112 has a field strength within a range that when the pulsed electric field is applied, pores 116 are formed in membranes 118 of cells 102A of the infected tissue 104.

The field strength of the pulsed electric field 112 may be, for example, about 40 kV/cm or less, about 30 kV/cm or less, and about 20 kV/cm or less. Additionally, the pulsed electric field 112 may be a plurality of electric field pulses 112 having a time duration of, for example, about 50 to 900 nanoseconds.

At 406 of the method, an antimicrobial agent 132 is applied into or onto the infected tissue 104 when the pulsed electric field 112 is applied. The antimicrobial agent 132 is operable to cross or integrate into the membrane 118 of the cells 102A when the pulsed electric field 112 is applied. The antimicrobial agent may be, for example, applied topically onto the infected tissue or may be applied by injecting into the infected tissue.

The antimicrobial agent 132 may include a member of the biguanide family. Such a member of the biguanide family may be, for example, Chlorhexidine.

At 408 of the method, at least one of an antibiotic, an antiseptic or an antifungal agent 134 is applied into or onto the infected tissue 104. The at least one of an antibiotic, an antiseptic or an antifungal agent 134 is operable to cross or integrate into the membrane 118 of the cells 102A when the pulsed electric field 112 is applied.

At 410 of the method, microorganisms (such as cells 102A) are inactivated via the at least of an antibiotic, an antiseptic or an antifungal agent 134 at a rate that is greater when the pulsed electric field 112 and antimicrobial agent 132 are applied simultaneously than when the pulsed electric field 112 and antimicrobial agent 132 are applied separately.

Referring to FIGS. 19-22, various additional examples of the applicator device 101 are illustrated and described.

Referring to FIGS. 19A-19C an embodiment of the applicator device 101 is depicted as including a double lead threaded combined device 1000 in accordance with an embodiment described herein. FIG. 19A shows a side view of the double lead combined device 1000 and its related components, and a detailed exploded side view of double lead combined 1000. FIG. 19B shows an exploded side view of the double lead combined device 1000 and its related components. FIG. 19C shows a side view of double lead combined device 1000 and related components.

The double lead combined device 1000 includes a double lead screw 1002 that may be comprised of any suitable non-conductive material. The double lead screw 1002 may, in one embodiment, be hollow, or may be solid, or may include one or more interior channels. The double lead screw 1002 may, for example, include one or more thread wires inside the double lead screw or outside the double lead screw. For example, the double lead screw 1002 may include a thread root wire 1004 and/or a thread lead 1 crest wire 1006 and/or a thread lead 2 crest wire 1008, and each of those wires may extend the length of the double lead screw 1002. The thread root wire 1004 and thread lead crest wires (1006, 1008) may be in a coil shape and may, in one embodiment, be comprised of titanium, stainless steel, or any combination thereof. In an example where the thread root wire and/or thread crest wires are in a coil shape, the coil shape may extend the length of the double lead screw 1002. The thread root wire 1004 may include a first thread root sub-wire 1004A and a second thread root sub-wire 1004B coiled around one another that form a repeating thread root wire cross-section 1005 where the first thread root sub-wire 1004A and a second thread root sub-wire 1004B meet while coiled around one another. The thread lead 1 crest wire 1006 and thread lead 2 1008 crest wire may in one embodiment each include one wire in a coil shape.

The double lead screw 1002 of the double lead combined 1000 may include ridges for placement of the thread root wire, thread lead 1 crest wire, and/or thread lead 2 crest wire. The ridges are shown, for example, as a thread crest 1 1010, a thread crest 2 1012, and a thread root 1015. The double lead screw 1002 may include one or more wire pathways 1014 for placement of the thread root wire 1004, thread lead 1 crest wire 1006, and/or thread lead 2 crest wire 1008. The wire pathway(s) 1014 may, in one embodiment, extend the length of the double lead screw 1002. The wire pathway(s) 1014 may, for example, be in a coil shape or in any suitable shape to accommodate the one or more wire (e.g., thread root wire 1004, thread lead 1 crest wire 1006, thread lead 2 crest wire 1008, and/or any additional useful wire).

The double lead screw 1002 of the double lead combined includes a first tip 1016 that is suitable for attachment to one or more wire connectors, including, for example, a root wire connector 1018 and a crest wire connector 1020. In one embodiment, the double lead screw 1002 may be attached to a root wire connector 1018 and a crest wire connector 1020. The double lead combined 1000 may, in one embodiment, be connected to a power supply via a cable or one or more wire connector. The one or more wire connector may extend from the double lead combined to a power source. Electrical connectors may be placed between the one or more screw wire and a harness to secure placement. The one or more electrical connector may be used to transfer electrical pulses to the one or more thread wires. The one or more thread wires are used to deliver electric pulses to a target subject.

Further included in this embodiment is one or more electrode array and optionally a plurality of electrodes. The electrode array may include a plurality of linear electrodes and the electrode array may be used for stimulating a pattern of electric current or voltage that is initially provided by a power source and may be used to deliver electrical pulses to a subject. In one embodiment, there is one electrode array, or alternatively, there may be two or more electrode arrays. The electrode array may be configured to provide effective administration of electrical pulses. The electrodes may, in one embodiment, have a diameter of between 0.5 millimeter and 2.0 millimeters. The electrodes may be independent of one another. The double lead combined may include one or more wires to facilitate transfer of electrical pulses from a power supply to the double lead combined, or may, alternatively, be wireless.

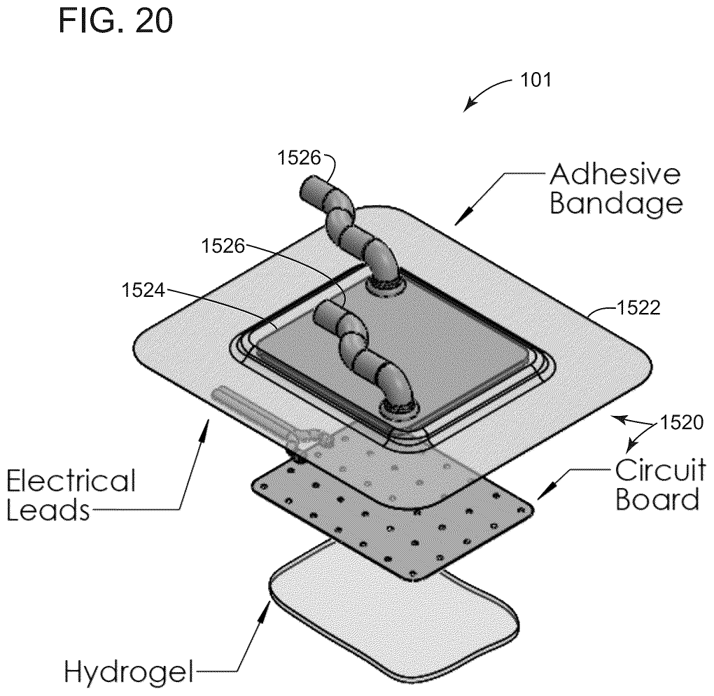

Referring to FIG. 20 an embodiment of applicator device 101 is depicted as including a negative pressure wound therapy device (NPWT device) 1520 in accordance with the present disclosure. FIG. 20 shows an exploded side view of the NPWT device 1520.

The NPWT device (also referred to herein as vacuum-assisted wound closure) includes a wound dressing system that continuously or intermittently applies sub-atmospheric pressure to the surface of a wound on a subject. The pressure applied may be used to pull and/or pump fluid, tissue, and/or cells from the subject. The NPWT in accordance with the present disclosure may include, for example, an open-pore polyurethane ether foam sponge, a semi-occlusive adhesive cover, a fluid collection system, and a suction pump.

An outer circumference 1522 of the NPWT device 1520 may contain an adhesive (e.g., glue, acrylate, vinyl resin, or other adhesive material) on a side that is placed onto a subject's skin. An inner circumference 1524 may contain as absorbent pad comprised of cotton, for example, and optionally, a thin, porous-polymer coating over the pad to keep it from sticking to the wound of a subject.

Inner circumference 1524 may include a circuit board and may further include a hydrogel. The inner circumference 1524 may further include one or more electrical leads, one or more electrodes, and may form one or more electrode array. The inner circumference may be in any suitable shape to facilitate placement of the circuit board, hydrogel, electrical leads, electrodes and/or, electrode array. As shown in FIG. 20, a two duct system 1526 is provided to apply sub-atmospheric pressure to the system, which provides a pressure to the surface of a wound on a subject.

The inner circumference 1524 may, in one embodiment, be connected to a power supply via a cable or one or more electrical lead 1530. The one or more electrical lead 1530 may extend from the inner circumference 1524 to a power source. Electrical connectors may be placed between the inner circumference 1524 and the electrical lead 1530 to secure placement. The one or more electrodes may be used to deliver electric pulses to a target subject. The NPWT may include one or more wires to facilitate transfer of electrical pulses from a power supply to the NPWT, or may, alternatively, be wireless.

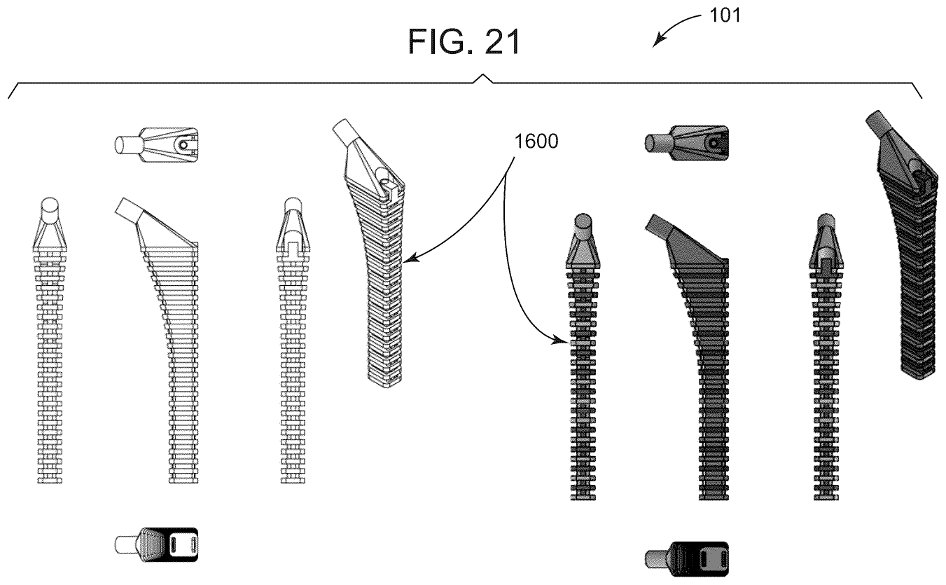

Referring to FIG. 21, an embodiment of applicator device 101 is depicted as including a side view of a hip stem 1600, in accordance with one embodiment described herein.

The hip stem 1600 may include a circuit board and may optionally include a hydrogel. The hip stem 1600 may further include one or more electrical leads, one or more electrodes, and may form one or more electrode array as shown in FIG. 21. The hip stem 1600 and electrode array may be in any suitable shape to facilitate placement of the hip stem 1600, circuit board, hydrogel, electrical leads, electrodes and/or, electrode array.

The hip stem 1600 may, in one embodiment, be connected to a power supply via a cable or one or more electrical lead. The one or more electrical lead may extend from the hip stem to a power source. Electrical connectors may be placed between the hip stem 1600 and the electrical lead to secure placement. The one or more electrodes may be used to deliver electric pulses to a target subject.

The hip stem 1600 may optionally include a plug that is removable and facilitates administration of electrical pulses and, in some embodiments, administration of one or more antimicrobial compound, antibiotic compound, antibody, biocidal agent, nanoparticles, self-assembling nanoparticles, viral particles, bacteriophage particles, bacteriophage DNA, genetic material, chemotherapy agent, growth factor, synthetic scaffold, natural scaffold, electrode, drug, microbe, bacteria, bioactive material, hydrogel, or any combination thereof. The hip stem 1600 may include one or more wires to facilitate transfer of electrical pulses from a power supply to the hip stem 1600, or may, alternatively, be wireless.

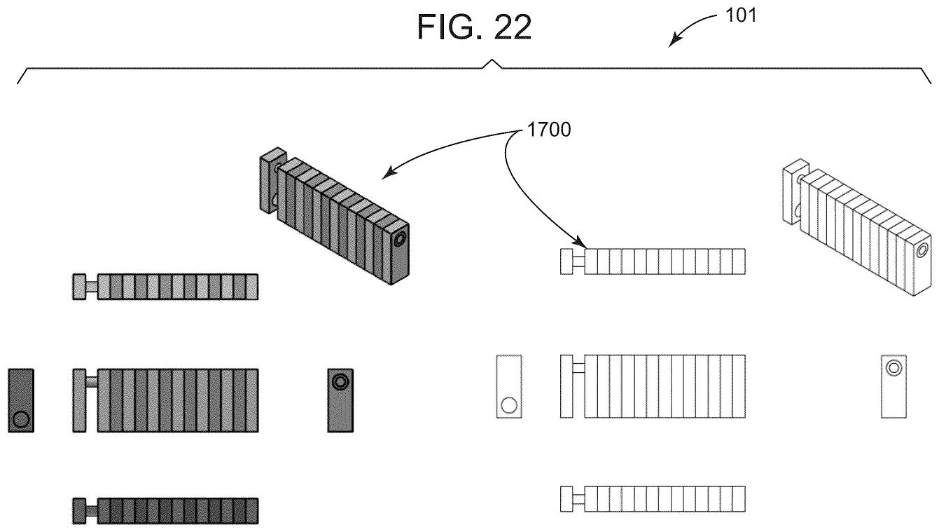

FIG. 22 shows an implant plate stack 1700 concept in accordance with one embodiment described herein.

The implant plate stack 1700 may include a circuit board and may optionally include a hydrogel. The implant plate stack 1700 may further include one or more electrical leads, one or more electrodes, and may form one or more electrode array as shown in FIG. 22. The implant plate stack 1700 and electrode array may be in any suitable shape to facilitate placement of the implant plate stack 1700, circuit board, hydrogel, electrical leads, electrodes and/or, electrode array.

The implant plate stack 1700 may, in one embodiment, be connected to a power supply via a cable or one or more electrical lead. The one or more electrical lead may extend from the implant plate stack to a power source. Electrical connectors may be placed between the implant plate stack 1700 and the electrical lead to secure placement. The one or more electrodes may be used to deliver electric pulses to a target subject.

The implant plate stack 1700 may be formed in any shape suitable for placement in an implant, including but not limited to, a hip, shoulder, ankle, spine or elbow implant. The implant plate stack 1700 may optionally include a plug that is removable and facilitates administration of electrical pulses and, in some embodiments, administration of one or more antimicrobial compound, antibiotic compound, antibody, biocidal agent, nanoparticles, self-assembling nanoparticles, viral particles, bacteriophage particles, bacteriophage DNA, genetic material, chemotherapy agent, growth factor, synthetic scaffold, natural scaffold, electrode, drug, microbe, bacteria, bioactive material, hydrogel, or any combination thereof. The implant plate stack 1700 may include one or more wires to facilitate transfer of electrical pulses from a power supply to the implant plate stack, or may, alternatively, be wireless.

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

Although the invention has been described by reference to specific examples, it should be understood that numerous changes may be made within the spirit and scope of the inventive concepts described. Accordingly, it is intended that the disclosure is not limited to the described examples, but that it has the full scope defined by the language of the following claims.

What is claimed is:

1. A system for treating infected tissue, the system comprising:
an applicator device comprising an electrode pair spaced apart and operable to be positioned against infected tissue; and
an electrical power supply operable to apply an electric field across the electrode pair to apply a pulsed electric field through the infected tissue, wherein the pulsed electric field has a field strength within a range that when the pulsed electric field is applied, pores are formed in membranes of cells of the infected tissue.

2. The system of claim 1, further comprising:
an antimicrobial agent operable to cross and/or integrate into the membrane of the cells when the electric field is applied.

3. The system of claim 2 comprising:
at least one of an antibiotic, an antiseptic or an antifungal agent operable to cross or integrate into the membrane of the cells when the electric field is applied.

4. The system of claim 3, wherein a microorganism inactivation rate of the at least one of an antibiotic, an antiseptic or an antifungal agent is greater when the pulsed electric field and antimicrobial agent are applied simultaneously than when the pulsed electric field and antimicrobial agent are applied separately.

5. The system of claim 2, wherein the antimicrobial agent comprises a member of the biguanide family.

6. The system of claim 2, wherein the antimicrobial agent comprises Chlorhexidine.

7. The system of claim 6, wherein the concentration of antimicrobial agent is between 0.001 percent and 2.0 percent.

8. The system of claim 1, further comprising:
a bridging resistor electrically connected in parallel with the electrode pair to the power supply, wherein the bridging resistor substantially matches an impedance of a pulse generator that the power supply is connected to.

9. The system of claim 1, wherein the electrodes are spaced apart at a distance of a range of about 0.1 millimeters (mm) to 5 mm.

10. The system of claim 1, wherein the field strength of the pulsed electric field is about 40 kilovolts per centimeter or less.

11. The system of claim 1, wherein the pulsed electric field is comprised of a plurality of electric field pulses having a time duration of about 50 to 900 nanoseconds.

12. The system of claim 1, wherein the applicator device comprises:
a flexible substrate operable to form a bandage for placement over the infected tissue; and
wherein the electrode pair are disposed on the substrate.

13. The system of claim 12 wherein the electrode pair comprise a plurality of parallel electrode pairs disposed on the substrate and extend longitudinally across the substrate.

14. The system of claim 13, wherein the substrate comprises a flexible printed circuit board that is operable to be electrically connected to the power supply.

15. The system of claim 1, wherein the applicator device comprises:
a wand having a handle and a head, wherein the electrode pair are disposed in a distal end of the head; and
wherein, the wand is operable to be grasped by the handle by a user and the distal end of the head is operable to be pressed by the user against the infected tissue.

16. The system of claim 15, wherein the electrode pair comprises a plurality of parallel electrode pairs spaced a substantially equal distance apart.

17. The system of claim 15, wherein the distal end of the head comprises at least one of a convex shape, a concave shape or a flat shape.

18. The system of claim 1, wherein the applicator device comprises a screw fastener configured to be threaded into the infected tissue.

19. The system of claim 18, wherein the infected tissue is a human bone.

20. The system of claim 18, wherein the applicator device comprises:
the screw fastener having threads that comprise a major diameter and a minor diameter; and
the pair of electrodes comprising a first electrode wrapped around the minor diameter and a second electrode wrapped around the major diameter.

21. The system of claim 1, wherein the applicator device comprises:
a pin operable to be inserted into the infected tissue; and
the pair of electrodes comprising a first electrode and a second electrode wrapped around an outside surface of a shaft of the pin, the first and second electrodes being at least one of evenly spaced apart along the shaft of the pin or parallel to each other along the shaft of the pin.

22. The system of claim 21, wherein the pin is hollow and has an inside surface, the inside surface defining a central channel, wherein the pin includes a plurality of spaced through holes disposed along the shaft of the pin, and wherein the through holes extend from the channel to the outside surface of the pin.

23. The system of claim 22, wherein the applicator device comprises:
a syringe operable to be connected to a distal end of the pin such that the syringe is in fluid communication with the channel, the syringe is operable to deliver fluids into the channel and out through the through holes.

24. The system of claim 23, wherein the fluids are at least one of an antimicrobial agent, antibiotic agent, an antifungal agent or an antiseptic agent.

25. The system of claim 1, wherein the applicator device comprises:
an implant that is operable to be inserted into the infected tissue; and
wherein the electrode pair are disposed on an outer surface of the implant.

26. The system of claim 25, wherein the electrode pair comprise a plurality of electrode pairs disposed on the outer surface of the implant.

27. The system of claim 25, wherein the tissue is human bone.

28. The system of claim 25, wherein the implant is one of a plate, a knee implant, a shoulder implant, an elbow implant, an ankle implant, a spine interbody implant or a hip stem.

29. The system of claim 1, wherein the applicator device comprise a negative pressure wound therapy device.

30. A system for treating infected tissue, the system comprising:
an applicator device comprising an electrode pair spaced apart and operable to be positioned against the infected tissue; and
an electrical power supply operable to generate an electric field having a field strength of 40 kV/cm or less across the electrode pair to apply a pulsed electric field being comprised of a plurality of electric field pulses having a time duration of about 50 to 900 nanoseconds through the infected tissue, wherein the pulsed electric field has a field strength within a range that when the pulsed electric field is applied, pores are formed in membranes of cells of the infected tissue; and
an antimicrobial agent operable to cross or integrate into the membrane of the cells when the pulsed electric field is applied.

31. The system of claim 30, comprising:
at least one of an antibiotic, an antiseptic or an antifungal agent operable to cross or integrate into the membrane of the cells when the pulsed electric field is applied;
wherein a microorganism inactivation rate of the at least one of an antibiotic, an antiseptic or an antifungal agent is greater when the pulsed electric field and antimicrobial agent are applied simultaneously than when the pulsed electric field and antimicrobial agent are applied separately.

32. The system of claim 30, wherein the pulsed electric field has an electric field strength of about 30 kV/cm or less.

33. The system of claim 30, wherein the antimicrobial agent comprises a member of the biguanide family.

34. The system of claim 30, wherein the antimicrobial agent comprises Chlorhexidine.

35. The system of claim 30, wherein the concentration of antimicrobial agent is between 0.001 percent and 2.0 percent.

36. The system of claim 30, wherein the applicator device comprises:
a flexible substrate operable to form a bandage for placement over the infected tissue; and
wherein the electrode pair are disposed on the substrate.

37. The system of claim 36 wherein the electrode pair comprise a plurality of parallel electrode pairs disposed on the substrate and extend longitudinally across the substrate.

38. The system of claim 30, wherein the applicator device comprises:
a wand having a handle and a head;
wherein the electrode pair are disposed in a distal end of the head; and
wherein, the wand is operable to be grasped by the handle by a user and the distal end of the head is configured to be pressed by the user against the infected tissue.

39. The system of claim 30, wherein the applicator device comprises:
a screw operable to be threaded into the infected tissue, the screw having threads that define a major diameter and a minor diameter; and wherein the pair of electrodes comprise a first electrode wrapped around the minor diameter and a second electrode wrapped around the major diameter.

40. The system of claim 30, wherein the applicator device comprises:
a pin operable to be inserted into the infected tissue; and
the pair of electrodes comprising a first electrode and a second electrode wrapped around an outside surface of the pin, the first and second thread wires being evenly spaced.

41. The system of claim 40, wherein the pin is hollow and has an inside surface, wherein the inside surface defines a central channel, the pin includes a plurality of spaced through holes disposed along a length of the pin, the through holes extend from the channel to the outside surface of the pin; and
a syringe connected to a distal end of the pin such that the syringe is in fluid communication with the channel, the syringe operable to deliver fluids into the channel and out from the through holes.

42. The system of claim 30, wherein the applicator device comprises:
an implant that is configured for insertion into the infected tissue; and
wherein the electrode pair are disposed on an outer surface of implant.

43. The system of claim 42, wherein the electrode pair comprise a plurality of electrode pairs disposed on the outer surface of the implant.

44. The system of claim 42, wherein the implant is at least one of a plate, a knee implant, a shoulder implant, an elbow implant, and ankle implant, a spine interbody implant or a hip stem.

45. The system of claim 30, wherein the applicator device comprise a negative pressure wound therapy device.

46. A method for treating infected tissue, the method comprising:
positioning an electrode pair against the infected tissue;
applying a pulsed electric field across the electrode pair generated from a power supply through the infected tissue, wherein the pulsed electric field has a field strength within a range that when the pulsed electric field is applied, pores are formed in membranes of cells of the infected tissue; and
applying an antimicrobial agent into or onto the infected tissue when the pulsed electric field is applied, wherein the antimicrobial agent crosses or integrates into the membrane of the cells when the pulsed electric field is applied.

47. The method of claim 46, comprising:
applying at least one of an antibiotic, an antiseptic or an antifungal agent into or onto the infected tissue, the at least one of an antibiotic, an antiseptic or an antifungal agent crosses or integrates into the membrane of the cells when the pulsed electric field is applied.

48. The method of claim 47, wherein a microorganism inactivation rate of the at least one of an antibiotic, an antiseptic or an antifungal agent is greater when the pulsed electric field and antimicrobial agent are applied simultaneously than when the pulsed electric field and antimicrobial agent are applied separately.

49. The method of claim 46, wherein the antimicrobial agent comprises a member of the biguanide family.

50. The method of claim 46, comprising:
spacing the electrode pair apart a distance within a range of 0.1 millimeters (mm) to 5 mm.

51. The method of claim 46, wherein the field strength is about 40 kilovolts per centimeter or less.

52. The method of claim 51, wherein the field strength is about 20 kilovolts per centimeter or less.

53. The method of claim 51, wherein the field strength is about 18 kilovolts per centimeter or less.

54. The method of claim 46, comprising:
applying the pulsed electric field as a plurality of electric field pulses having a time duration of about between 50 to 900 nanoseconds.

55. The method of claim 46, wherein the electrode pair is disposed within an applicator device.

56. The method of claim 55, wherein the applicator device comprises at least one of a wand, a pin, a screw, a bandage, a negative pressure wound therapy device or an implant.
